(19) United States
(12) Patent Application Publication
(10) Pub. No.: US 2022/0202672 A1
(43) Pub. Date: Jun. 30, 2022

(54) BABY LOTION

(71) Applicant: Melaleuca, Inc., Idaho Falls, ID (US)

(72) Inventor: Rebecca L. Zehntner, Blackfoot, ID (US)

(21) Appl. No.: 17/565,944

(22) Filed: Dec. 30, 2021

Related U.S. Application Data
(60) Provisional application No. 63/133,053, filed on Dec. 31, 2020.

(51) Int. Cl. A61K 8/41; A61K 8/33; A61K 8/37; A61K 8/9789; A61K 8/34; A61K 8/9794; A61K 8/67; A61Q 19/00

(52) U.S. Cl. CPC A61K 8/416; A61K 8/33; A61K 8/37; A61Q 19/007; A61K 8/345; A61K 8/9794; A61K 8/678; A61K 8/9789

(57) ABSTRACT

Materials and methods for formulating skin care compositions suitable for use on babies and toddlers are provided herein. For example, this document provides materials and methods for formulating lotion compositions that contain natural ingredients, are not irritating to babies' skin, and can be used to moisturize and soothe dry skin.

### CROSS-REFERENCE TO RELATED APPLICATIONS

This application claims benefit of priority from U.S. Provisional Application Ser. No. 63/133,053, filed on Dec. 31, 2020. The disclosure of the prior application is considered part of (and is incorporated by reference in) the disclosure of this application.

### TECHNICAL FIELD

This document relates to materials and methods for formulating skin care compositions suitable for use on babies and toddlers. For example, this document relates to materials and methods for formulating lotion compositions that contain natural ingredients, are not irritating to babies' skin, and can be used to moisturize and soothe dry skin.

### BACKGROUND

Young children, including babies and toddlers, typically have sensitive, delicate skin. Skin care products (e.g., lotions and creams) designed for adult use often are not suitable for use with small children.

### SUMMARY

This document is based, at least in part, on the development of compositions that are suitable for use on young children. The compositions provided herein contain natural and safe ingredients, resulting in products that are low (or non-) irritating to babies' skin. In addition, the compositions provided herein can be used to reduce dry skin, increase smoothness and moisturization of skin, and reduce skin irritation.

In a first aspect, this document features a skin care composition that contains distearyldimonium chloride, dicaprylyl ether, oleyl erucate, Glycine soya oil, a Calendula officinalis extract, hydrogenated cottonseed oil, and glycerin. The composition can contain from about 0.05 to about 50% w/w distearyldimonium chloride. The composition can contain from about 0.15 to about 15 w/w dicaprylyl ether. The composition can contain from about 0.15 to about 15 w/w oleyl erucate. The composition can contain from about 0.5 to about 50% w/w G. soya oil. The composition can contain from about 0.00125 to about 0.125% w/w C. officinalis extract. The composition can further contain tocopherol. The composition can contain from about 0.00005 to about 0.005 w/w tocopherol. The composition can contain from about 0.075 to 7.5 w/w hydrogenated cottonseed oil. The composition can contain from about 1 to about 50% w/w glycerin. The composition can further contain cetyl alcohol. The composition can contain from about 0.3 to about 30% w/w cetyl alcohol. The composition can further contain polyglyceryl-6 distearate. The composition can contain from about 0.1 to about 10% w/w polyglyceryl-6 distearate. The composition can further contain colloidal oatmeal. The composition can contain from about 0.1 to about 10% w/w colloidal oatmeal. The composition can further contain benzyl alcohol. The composition can contain from about 0.05 to about 5 w/w benzyl alcohol. The composition can further contain jojoba esters. The composition can contain from about 0.035 to about 3.5 w/w jojoba esters. The composition can further contain sodium benzoate. The composition can contain from about 0.016 to about 1.6% w/w sodium benzoate. The composition can further contain polyglyceryl-3 beeswax. The composition can contain from about 0.014 to about 1.4% w/w polyglyceryl-3 beeswax. The composition can further contain potassium sorbate. The composition can contain from about 0.008 to about 0.8% w/w potassium sorbate. The composition can further contain trisodium ethylenediamine disuccinate. The composition can contain from about 0.005 to about 0.5 w/w trisodium ethylenediamine disuccinate. The composition can further contain sodium chloride. The can contain composition can contain from about 0.005 to about 0.5 w/w sodium chloride. The composition can further contain one or more fragrance compounds. The composition can contain from about 0.07 to about 7% w/w of said one or more fragrance compounds. The composition can have a viscosity of about 1×10⁵ to about 7×10⁵ centipoise. The composition can be formulated as a lotion or cream.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This document provides compositions containing ingredients that are safe for use on young children. The compositions provided herein are low irritating (e.g., non-irritating) and hypoallergenic, and typically contain multiple natural (plant-derived) ingredients such as glycerin, Calendula officinalis flower extract, and Glycine soya oil. Moreover, the compositions provided herein do not contain petrolatum, which is sometimes used as a barrier or moisturizing agent.

The compositions provided herein can contain any appropriate ingredients, including, without limitation, one or more surfactants, humectants, moisturizers, preservatives, fragrances, pH adjustors, chelating agents, emollients, skin conditioning agents, thickening agents, flow agents, emulsifiers, and stabilizers. Examples of components that can be included in the compositions provided herein are as follows.

Distearyldimonium chloride is a plant-derived skin conditioner and emulsifier. In some cases, the compositions provided herein can contain distearyldimonium chloride in an amount from about 0.5 to about 50% w/w (e.g., from about 0.5 to about 2.5% w/w, from about 2.5 to about 5 w/w, from about 5 to about 10% w/w, from about 10 to about 25% w/w, or from about 25 to about 50% w/w). In some cases, distearyldimonium chloride can be present in a composition provided herein in an amount from about 2.5 to about 7.5% w/w (e.g., from about 4 to about 6% w/w).

Dicaprylyl ether is a plant-derived skin conditioning agent and emollient that can be plant-derived. In some cases, the compositions provided herein can contain dicarpylyl ether in an amount from about 0.15 to about 15% w/w (e.g., from about 0.15 to about 0.5% w/w, from about 0.5 to about 3% w/w, from about 3 to about 5% w/w, from about 5 to about 10% w/w, or from about 10 to about 15% w/w). In some cases, dicaprylyl ether can be present in a composition provided herein in an amount from about 0.75 to about 2.25% w/w (e.g., from about 1.2 to about 1.8% w/w).

Oleyl erucate is another plant-derived skin conditioning agent and emollient. In some cases, the compositions provided herein can contain oleyl erucate in an amount from about 0.15 to about 15% w/w (e.g., from about 0.15 to about 0.5% w/w, from about 0.5 to about 3% w/w, from about 3 to about 5% w/w, from about 5 to about 10% w/w, or from about 10 to about 15% w/w). In some cases, oleyl erucate can be present in a composition provided herein in an amount from about 0.75 to about 2.25% w/w (e.g., from about 1.2 to about 1.8% w/w).

Glycine soya oil is a plant-derived skin conditioning agent that can help to moisturize and soften skin. In some cases, the compositions provided herein can contain G. soya oil in an amount from about 0.5 to about 50% w/w (e.g., from about 0.5 to about 1% w/w, from about 1 to about 3% w/w, from about 3 to about 5% w/w, from about 5 to about 10% w/w, from about 10 to about 30% w/w, or from about 30 to about 50% w/w). In some cases, G. soya oil can be present in a composition provided herein in an amount from about 3 to about 9% w/w (e.g., from about 5 to about 7% w/w).

Calendula officinalis flower extract (also referred to as Calendula oil) is a moisturizer and emollient that can condition and soothe the skin. In some cases, the compositions provided herein can contain a C. officinalis extract in an amount from about 0.00125 to about 0.125% w/w (e.g., from about 0.00125 to about 0.005% w/w, from about 0.005 to about 0.01% w/w, from about 0.01 to about 0.05% w/w, from about 0.05 to about 0.1% w/w, or from about 0.1 to about 0.125% w/w). In some cases, a C. officinalis flower extract can be present in a composition provided herein in an amount from about 0.006 to about 0.02% w/w (e.g., from about 0.001 to about 0.0015% w/w).

Tocopherol is an emollient and skin conditioning agent. In some cases, the compositions provided herein can contain tocopherol in an amount from about 0.00005 to about 0.005% w/w (e.g., from about 0.00005 to about 0.0001% w/w, from about 0.0001 to about 0.0005% w/w, from about 0.0005 to about 0.001% w/w, or from about 0.001 to about 0.005% w/w). In some cases, tocopherol can be present in a composition provided herein in an amount from about 0.00025 to about 0.00075% w/w (e.g., from about 0.0004 to about 0.0006% w/w).

Hydrogenated cottonseed oil is a plant-derived oil that can moisturize, condition, and soften skin. In some cases, the compositions provided herein can contain hydrogenated cottonseed oil in an amount from about 0.075 to about 7.5% w/w (e.g., from about 0.075 to about 0.5% w/w, from about 0.5 to about 1% w/w, from about 1 to about 3% w/w, or from about 3 to about 7.5% w/w). In some cases, hydrogenated cottonseed oil can be present in a composition provided herein in an amount from about 0.38 to about 1.1% w/w (e.g., from about 0.6 to about 0.9% w/w).

Glycerin is a humectant and skin conditioning agent that can draw moisture into the skin, and can be plant-derived. In some cases, the compositions provided herein can contain glycerin in an amount from about 1 to about 50% w/w (e.g., from about 1 to about 3% w/w, from about 3 to about 5% w/w, from about 5 to about 10% w/w, from about 10 to about 15% w/w, from about 15 to about 25% w/w, or from about 25 to about 50% w/w). In some cases, glycerin can be present in a composition provided herein in an amount from about 6 to about 18% w/w (e.g., from about 9 to about 15 w/w).

Cetyl alcohol is an emulsifier that can be plant-derived. In some cases, the compositions provided herein can contain cetyl alcohol in an amount from about 0.3 to about 30% w/w (e.g., from about 0.3 to about 1% w/w, from about 1 to about 3% w/w, from about 3 to about 5% w/w, from about 5 to about 15% w/w, or from about 15 to about 30% w/w). In some cases, cetyl alcohol can be present in a composition provided herein in an amount from about 1.5 to about 4.5% w/w (e.g., from about 2 to about 4% w/w).

Polyglyceryl-6 distearate is another emulsifier that can be plant-derived. In some cases, the compositions provided herein can contain polyglyceryl-6 distearate in an amount from about 0.1 to about 10% w/w (e.g., from about 0.1 to about 0.5% w/w, from about 0.5 to about 1% w/w, from about 1 to about 3% w/w, or from about 3 to about 10% w/w). In some cases, polyglyceryl-6 distearate can be present in a composition provided herein in an amount from about 0.7 to about 2.1% w/w (e.g., from about 1.1 to about 1.7% w/w).

Polyglyceryl-3 beeswax also is an emulsifier that can be plant-derived. In some cases, the compositions provided herein can contain polyglyceryl-3 beeswax in an amount from about 0.014 to about 1.4% w/w (e.g., from about 0.014 to about 0.1% w/w, from about 0.1 to about 0.2% w/w, from about 0.2 to about 0.7% w/w, or from about 0.7 to about 1.4% w/w). In some cases, polyglyceryl-6 distearate can be present in a composition provided herein in an amount from about 0.07 to about 0.21% w/w (e.g., from about 0.12 to about 0.16% w/w).

Jojoba esters are plant-derived stabilizers and thickening agents that also can serve as moisturizers. In some cases, the compositions provided herein can contain jojoba esters in an amount from about 0.035 to about 3.5% w/w (e.g., from about 0.035 to about 0.1% w/w, from about 0.1 to about 0.3% w/w, from about 0.3 to about 0.5% w/w, from about 0.5 to about 2% w/w, or from about 2 to about 3.5% w/w). In some cases, jojoba esters can be present in a composition provided herein in an amount from about 0.17 to about 0.51% w/w (e.g., from about 0.2 to about 0.4% w/w).

Colloidal oatmeal is a skin conditioner and soother. In some cases, the compositions provided herein can contain colloidal oatmeal in an amount from about 0.1 to about 10% w/w (e.g., from about 0.1 to about 0.5% w/w, from about 0.5 to about 1% w/w, from about 1 to about 3% w/w, or from about 3 to about 10% w/w). In some cases, colloidal oatmeal can be present in a composition provided herein in an amount from about 0.5 to about 1.5% w/w (e.g., from about 0.8 to about 1.2% w/w).

Benzyl alcohol is a preservative. In some cases, the compositions provided herein can contain benzyl alcohol in an amount from about 0.05 to about 5% w/w (e.g., from about 0.05 to about 0.1% w/w, from about 0.1 to about 0.5% w/w, from about 0.5 to about 1% w/w, from about 1 to about 3% w/w, or from about 3 to about 5% w/w). In some cases, benzyl alcohol can be present in a composition provided herein in an amount from about 0.25 to about 0.75% w/w (e.g., from about 0.4 to about 0.6% w/w).

Sodium benzoate is a preservative. In some cases, the compositions provided herein can contain sodium benzoate in an amount from about 0.016 to about 1.6% w/w (e.g., from about 0.016 to about 0.05% w/w, from about 0.05 to about 0.1% w/w, from about 0.1 to about 0.2% w/w, from about 0.2 to about 1% w/w, or from about 1 to about 1.6% w/w). In some cases, sodium benzoate can be present in a composition provided herein in an amount from about 0.08 to about 0.24% w/w (e.g., from about 0.12 to about 0.2% w/w).

Potassium sorbate also is a preservative. In some cases, the compositions provided herein can contain potassium sorbate in an amount from about 0.008 to about 0.8% w/w (e.g., from about 0.008 to about 0.05% w/w, from about 0.05 to about 0.1% w/w, from about 0.1 to about 0.3% w/w, from about 0.3 to about 0.5% w/w, or from about 0.5 to about 0.8% w/w). In some cases, potassium sorbate can be present in a composition provided herein in an amount from about 0.04 to about 0.12% w/w (e.g., from about 0.07 to about 0.09% w/w).

Trisodium ethylenediamine disuccinate is a plant-derived chelating agent. In some cases, the compositions provided herein can contain trisodium ethylenediamine disuccinate in an amount from about 0.005 to about 0.5% w/w (e.g., from about 0.005 to about 0.01% w/w, from about 0.01 to about 0.05% w/w, from about 0.05 to about 0.1% w/w, from about 0.1 to about 0.3% w/w, or from about 0.3 to about 0.5% w/w). In some cases, trisodium ethylenediamine disuccinate can be present in a composition provided herein in an amount from about 0.03 to about 0.09% w/w (e.g., from about 0.05 to about 0.07% w/w).

Sodium chloride can serve as a stabilizer. In some cases, the compositions provided herein can contain sodium chloride in an amount from about 0.005 to about 0.5% w/w (e.g., from about 0.005 to about 0.01% w/w, from about 0.01 to about 0.05% w/w, from about 0.05 to about 0.1% w/w, from about 0.1 to about 0.3% w/w, or from about 0.3 to about 0.5% w/w). In some cases, sodium chloride can be present in a composition provided herein in an amount from about 0.025 to about 0.075% w/w (e.g., from about 0.04 to about 0.06% w/w).

In some cases, the compositions provided herein also can contain one or more fragrance compounds (e.g., one or more plant-based fragrance compounds). In some cases, the compositions provided herein can contain one or more fragrance compounds in an amount from about 0.07 to about 7% w/w (e.g., from about 0.07 to about 0.1% w/w, from about 0.1 to about 0.5% w/w, from about 0.5 to about 1% w/w, from about 1 to about 4% w/w, or from about 4 to about 7% w/w). In some cases, fragrance compounds can be present in a composition provided herein in an amount from about 0.35 to about 1% w/w (e.g., from about 0.6 to about 0.8% w/w).

In addition, the compositions provided herein can include water, which can make up the remainder of the composition apart from the other ingredients. In some cases, the compositions provided herein can contain water in an amount from about 30 to about 95% w/w (e.g., from about 30 to about 40% w/w, from about 40 to about 50% w/w, from about 50 to about 60% w/w, from about 60 to about 70% w/w, from about 70 to about 80% w/w, or from about 80 to about 95% w/w). In some cases, water can be present in a composition provided herein in an amount from about 33 to about 99% w/w (e.g., about 50 to about 80% w/w).

Any appropriate combination of the ingredients listed above can be used in the compositions provided herein. In some cases, for example, a composition can include distearyldimonium chloride, dicaprylyl ether, oleyl erucate, Glycine soya oil, a Calendula officinalis extract, tocopherol, hydrogenated cottonseed oil, and glycerin. In addition, in some cases, a composition provided herein also can include any one or more of the other ingredients listed above. Moreover, a composition provided herein can further include any other appropriate ingredient (e.g., any other suitable surfactant, humectant, moisturizer, preservative, fragrance, pH adjustor, chelating agent, emollient, stabilizer, skin conditioning agent, thickening agent, or flow agent).

In some cases, any of the above ingredients can be substituted with any other appropriate ingredient. For example, the hydrogenated cottonseed oil and/or Glycine soya oil can be replaced by Ricinus commuis (castor) seed oil, hydrogenated castor oil, and Copernicia cerifera (carnauba) wax; vegetable oil; or canola oil, Ricinus communis (castor) seed oil, polyhydroxystearic acid, Euphorbia cerifera (candililla) wax, Helianthus annuus (sunflower) seed wax, and tocopheryl acetate.

The compositions described herein can have any appropriate pH. In general, the pH of the compositions is such that they are low irritating (e.g., non-irritating) to the skin or eyes. In some cases, a composition provided herein can have a pH of about 5 to about 6 (e.g., from about 5.0 to about 5.3, from about 5.2 to about 5.5, from about 5.4 to about 5.7, or from about 5.7 to about 6.0).

The compositions provided herein also can have any appropriate viscosity. In some cases, a lotion composition provided herein can have a viscosity from about 1×10⁵ to about 7×10⁵ centipoise (cps) (e.g., from about 1×10⁵ to about 3×10⁵ cps, from about 3×10⁵ to about 5×10⁵ cps, or from about 5×10⁵ to about 7×10⁵).

The invention will be further described in the following example, which does not limit the scope of the invention described in the claims.

### EXAMPLE

A concept study was conducted through an online survey to assess the attitudes and usage of more than 280 mothers of children aged two years and under. Children of the survey participants were about 50:50 boy:girl, and about 50:50 baby:toddler. Participants indicated that they were open to using a natural baby care product or had tried using a natural baby care product in the past.

A baby lotion composition was prepared according to the formulation in TABLE 1, and was tested in a blind consumer study of 91 participants selected from the concept study based at least in part on their willingness to test the product for a two week period. Participants completed a survey about 40 minutes after placing the lotion on their child's leg(s). More than 95% of the participants stated that the composition was gentle on their children's skin, and about 90% of participants stated that the composition left their child's skin feeling nourished and provided relief from dry skin. Similar results were obtained when the composition was pediatrician and dermatologist tested.

TABLE 1

| | Active amount |
|---|---|
| Ingredient | (% w/w) |
| water | 33-99 |
| glycerin | 6-18 |
| Glycine soja (soybean) oil | 3-9 |
| distearyldimonium chloride | 2.5-7.5 |
| cetyl alcohol | 1.5-4.5 |
| oleyl erucate | 0.75-2.25 |
| dicaprylyl ether | 0.75-2.25 |
| polyglyceryl-6 distearate | 0.7-2.1 |
| colloidal oatmeal | 0.5-1.5 |
| hydrogenated cottonseed oil | 0.38-1.1 |
| fragrance | 0.35-1 |
| benzyl alcohol | 0.25-0.75 |
| jojoba esters | 0.17-0.51 |
| sodium benzoate | 0.08-0.24 |
| polyglyceryl-3 beeswax | 0.07-0.21 |
| potassium sorbate | 0.04-0.12 |
| trisodium ethylenediamine disuccinate | 0.03-0.09 |
| sodium chloride | 0.025-0.075 |
| Calendula officinalis flower extract | 0.006-0.02 |
| tocopherol | 0.00025-0.00075 |

In addition, dermal evaluation scores for erythema, edema, and dryness were obtained for the children (babies or toddlers) of 30 study participants at baseline and after 4 weeks of at least once daily use of the composition. Results are shown in TABLE 2. Corneometer measurements also were taken to assess the hydration level of the superficial skin layers for 30 of the children at baseline, 4 hours after application, and 24 hours after application at least once daily over the course of one week. As shown in TABLE 3, the composition significantly increased skin hydration levels.

TABLE 2

| | | Frequency (average of right and left legs) | | | | | |
|---|---|---|---|---|---|---|---|
| Assessment | Time point | N | 0* | 0.5 | 1 | 2 | 3 |
| Erythema | Baseline | 30 | 30 | 0 | 0 | 0 | 0 |
| | Week 4 | 30 | 30 | 0 | 0 | 0 | 0 |
| Edema | Baseline | 30 | 30 | 0 | 0 | 0 | 0 |
| | Week 4 | 30 | 30 | 0 | 0 | 0 | 0 |
| Dryness | Baseline | 30 | 22 | 0 | 8 | 0 | 0 |
| | Week 4 | 30 | 30 | 0 | 0 | 0 | 0 |

*Dermal scoring: 0 = None 1 = Slight 2 = Mild 3 = Moderate 4 = Severe

TABLE 3

| Mean corneometer measurements | | | | Mean change from baseline | | |
|---|---|---|---|---|---|---|
| | 4 hours ± | 24 hours ± | | 4 hours ± | 24 hours ± | |
| | 30 min | 30 min | | 30 min | 30 min | |
| Baseline | post-appln | post-appln | Week 1 | post-appln | post-appln | Week 1 |
| 22.5 | 27.3 | 26.8 | 27.1 | 4.8 | 4.3 | 4.4 |
| | | | | (p < 0.0001) | (p < 0.0001) | (p < 0.0001) |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A skin care composition comprising:
distearyldimonium chloride,
dicaprylyl ether,
oleyl erucate,
Glycine soya oil,
a Calendula officinalis extract,
hydrogenated cottonseed oil, and
glycerin.

2. The skin care composition of claim 1, wherein said composition comprises from about 0.05 to about 50% w/w distearyldimonium chloride.

3. The skin care composition of claim 1, wherein said composition comprises from about 0.15 to about 15% w/w dicaprylyl ether.

4. The skin care composition of claim 1, wherein said composition comprises from about 0.15 to about 15% w/w oleyl erucate.

5. The skin care composition of claim 1, wherein said composition comprises from about 0.5 to about 50% w/w G. soya oil.

6. The skin care composition of claim 1, wherein said composition comprises from about 0.00125 to about 0.125% w/w C. officinalis extract.

7. The skin care composition of claim 1, wherein said composition comprises from about 0.075 to 7.5% w/w hydrogenated cottonseed oil.

8. The skin care composition of claim 1, wherein said composition comprises from about 1 to about 50% w/w glycerin.

9. The skin care composition of claim 1, further comprising cetyl alcohol.

10. The skin care composition of claim 9, wherein said composition comprises from about 0.3 to about 30% w/w cetyl alcohol.

11. The skin care composition of claim 1, further comprising polyglyceryl-6 distearate.

12. The skin care composition of claim 11, wherein said composition comprises from about 0.1 to about 10% w/w polyglyceryl-6 distearate.

13. The skin care composition of claim 1, further comprising colloidal oatmeal.

14. The skin care composition of claim 13, wherein said composition comprises from about 0.1 to about 10% w/w colloidal oatmeal.

15. The skin care composition of claim 1, further comprising benzyl alcohol.

16. The skin care composition of claim 15, wherein said composition comprises from about 0.05 to about 5% w/w benzyl alcohol.

17. The skin care composition of claim 1, further comprising jojoba esters.

18. The skin care composition of claim 17, wherein said composition comprises from about 0.035 to about 3.5% w/w jojoba esters.

19. The skin care composition of claim 1, further comprising sodium benzoate.

20. The skin care composition of claim 19, wherein said composition comprises from about 0.016 to about 1.6% w/w sodium benzoate.

21. The skin care composition of claim 1, further comprising polyglyceryl-3 beeswax.

22. The skin care composition of claim 21, wherein said composition comprises from about 0.014 to about 1.4% w/w polyglyceryl-3 beeswax.

23. The skin care composition of claim 1, further comprising potassium sorbate.

24. The skin care composition of claim 23, wherein said composition comprises from about 0.008 to about 0.8% w/w potassium sorbate.

25. The skin care composition of claim 1, further comprising trisodium ethylenediamine disuccinate.

26. The skin care composition of claim 25, wherein said composition comprises from about 0.005 to about 0.5% w/w trisodium ethylenediamine disuccinate.

27. The skin care composition of claim 1, further comprising sodium chloride.

28. The skin care composition of claim 27, wherein said composition comprises from about 0.005 to about 0.5% w/w sodium chloride.

29. The skin care composition of claim 1, further comprising tocopherol.

30. The skin care composition of claim 29, wherein said composition comprises from about 0.00005 to about 0.005% w/w tocopherol.

31. The skin care composition of claim 1, further comprising one or more fragrance compounds.

32. The skin care composition of claim 31, wherein said composition comprises from about 0.07 to about 7% w/w of said one or more fragrance compounds.

33. The skin care composition of claim 1, wherein said composition has a viscosity of about 1×10⁵ to about 7×10⁵ centipoise.

34. The skin care composition of claim 1, wherein said composition is formulated as a lotion or cream.
